(19) United States
(12) Patent Application Publication
(10) Pub. No.: US 2004/0053867 A1
(43) Pub. Date: Mar. 18, 2004

(54) Materials and methods relating to a novel splice variant of a na+ dependent glutamate transporter

(76) Inventors: Deborah Mason, Hereford (GB); Jim Francis Huggett, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/240,024

(22) PCT Filed: Mar. 26, 2001
(86) PCT No.: PCT/GB01/01360

(30) Foreign Application Priority Data
Mar. 29, 2000 (GB) 0007632.3
Nov. 22, 2000 (GB) 0028503.1

(51) Int. Cl. A61K 48/00; C12Q 1/68; C07H 21/04; C07K 14/705

(52) U.S. Cl. 514/44; 435/6; 435/320.1; 435/69.1; 435/325; 530/350; 536/23.5

(57) ABSTRACT

The invention relates to the Na+ dependent glutamate transporter GLAST-1 and a splice variant thereof. A novel splice variant has been bound (GLAST-1a) which lacks exon 3 resulting in a loss of about 46 amino acids. The protein is altered in such a way that indicates altered function of the transporter. Indeed, the inventors have surprisingly determined that the splice variant has a reversed transport direction as compared to GLAST-1. Thus, the invention provides materials and methods relating to the splice variant GLAST-1a including the amino acid and nucleic acid sequence; materials and methods relating to the detection in vivo or in vitro of the GLAST-1a; and materials and methods relating to the modulation of excitatory amino acids (EAAS) signalling.

### FIELD OF THE INVENTION

The present invention concerns materials and methods relating to a novel splice variant of a Na⁺ dependent glutamate transporter. Particularly, but not exclusively, the present invention concerns the Na⁺ dependent glutamate transporter GLAST-1 and a splice variant thereof.

### BACKGROUND TO THE INVENTION

Bone mass is adjusted according to local and systemic influences. This process, called remodelling, allows the repair of damaged tissue and maintenance of optimal bone matrix integrity. There are a number of pathologies that have been linked to a breakdown in the remodelling cycle, the most common being osteoporosis. While the bone remodelling cycle is well characterised its control is poorly understood. Glutamate signalling, more commonly associated with the central nervous system, has recently been implicated as a possible mechanism by which bone cells might communicate in response to their mechanical environment. This was demonstrated by the discovery that an mRNA with homology to GLAST-1 is down regulated by mechanical loading of osteocytes in-vivo (Mason. 1997).

In the central nervous system (CNS) GLAST-1 is a Na⁺ dependant symport of glutamate and aspartate that transports the excitatory amino acid from the nerve synapse back into the neuron directly after neurotransmission. This results in the termination of neurotransmission, removal of potentially toxic excitatory amino acids (EAA) from the synapse and recycling it for re-use. There are many studies investigating the structure of GLAST-1 and the other Na⁺ dependent excitatory amino acid transporters. It is agreed that GLAST-1 has six transmembrane α-helices at the N-terminal, the C-terminal however is less clear. Wahle et al hypothesise that the C-terminal of GLAST-1 is composed of four transmembrane β sheets (Wahle. 1996). More recently it has been proposed that this region is composed of another α-helix N terminal of a loop pore which is followed by 2 hydrophobic regions that do not span the membrane and a final TM α helix (Seal. 2000). A recent review of all the known Na⁺-dependent EAA transporters favours the hypothesis that there is a re-enterent pore, as opposed to β-sheets, in the C-terminal although there is still disagreement on the flanking transmembrane domains (Slotboom. 1999).

The role of GLAST-1, a Na⁺ dependent transporter of excitatory amino acids (EAA), has been extensively studied in the rat central nervous system (CNS). GLAST-1 is a member of a family EAA transporters which are responsible for terminating the signal across the nerve synapse. This is achieved by transporting EAA into cells at the synayse thus removing potentially toxic EAA from the synapse and “recycling” EAA for further signalling.

Previously, during an experiment designed to isolate genes involved in osteogenesis in vivo, the present inventors identified GLAST-1 as a candidate (Mason, 1997). Further investigation by RT-PCR revealed an mRNA which possessed exons 2, 3 and 4 of GLAST-1 expressed in rat bone suggesting a potential role for EAA signalling in communication between bone cells. The restriction of EAA signalling to the CNS has been previously questioned when GLAST-1 mRNA expression was reported in other tissues (Tanaka, 1993). The findings of Mason et al (Mason, 1997) further questioned this notion.

### SUMMARY OF THE INVENTION

One of the present inventors previously found different sized GLAST mRNAs in bone and brain suggesting that this gene is expressed as a number of splice variants. However, through further investigations, the present inventors have surprisingly found a novel splice variant of the GLAST-1 gene where exon 3 is removed resulting in the loss of 46 amino acids. This novel splice variant has been termed GLAST-1a. The inventors have further discovered that sequence of this novel protein is altered in a way that indicates altered function of the transporter. In fact, the inventors believe that the altered sequence results in this splice variant protein have a reversed transport direction as opposed to the GLAST-1 protein.

This discovery has a number of important and industrially applicable implications, particularly with regard to modulating excitatory amino acids (EAAs) signals in disease. There is much evidence that transporters that modulate EAA levels are important in a wide range of diseases. Such diseases include those resulting from abnormal EAA levels and/or altered mechanical environment. It is well known that GLAST-1 transports EAAs during normal neurotransmission in the CNS and is involved in disorders of the CNS where levels are disrupted such as epilepsy, Alziemers Disease, Parkinsons Disease, stroke, trauma, dementia and neurotoxicity due to ischaemia and anoxia ([1,2], Obrenovitch 1996). The inventors have shown for the first time that GLAST-1a is expressed at the mRNA and protein level in brain tissue [3] and may therefore be used to treat disorders of the CNS. Recently, it has been shown that GLAST-1 is expressed in cells of the retina and that the elevated levels of glutamate associated with glaucoma are accompanied by reduced expression of GLAST-1 [4]. The role of GLAST-1 in the eye has been elucidated using either antisense knock out or pharmacologic inhibition of GLAST-1 in retinal ganglion cells. These studies shows that inactivation of GLAST-1 resulted in elevated extracellular glutamate levels and retinal excitotoxicity [5]. The inventors also have recently detected GLAST-1a expression in rat retinal cDNA which is supportive of a role for this variant in diseases of the eye such as glaucoma.

There is now good evidence for glutamate signalling in bone with the discovery of functional metabotropic [6] and NMDA receptors [7] in osteoblasts as well as other components of glutamate signalling in bone cells [8-11]. In addition, in vitro studies suggest that glutamate affects osteoblast and osteoclast differentiation and activity [8] [12]. The inventors have preliminary data suggesting that extracellular glutamate concentration affects the levels of expression of GLAST-1 and GLAST-1a mRNA (Huggett, Mustafa and Mason unpublished data) and other workers have shown that glutamate concentration can affect gap junction formation in osteoblasts [13]. These data along with the inventors' evidence that GLAST-1a mRNA and protein is expressed by bone cells in vivo [3] indicate that GLAST-1a may be used in treatment of disorders of bone.

Recent data show that inflammation of synovial joints is associated with elevated glutamate levels both in patients presenting with arthritis [14] and in animal models of inflammation [15, 16]. The inventors have shown GLAST-1 and GLAST-1a mRNA expression in many of the cell types present in the joint (Huggett and Mason unpublished data). This indicates that glutamate signalling may be important in the inflammatory response and that GLAST-1a may be used in the treatment of such conditions, in particular those associated with the arthritides.

Components of glutamate signalling are also expressed by keratinocytes [17] and glutamate levels are elevated in wound fluid {18} which indicates that glutamate signalling may be important in epidermal repair. As a component of this signalling mechanism, GLAST-1a may also be used in treatment of disorders of the skin and wound healing. In addition to bone and brain, the inventors have also detected GLAST-1 and/or GLAST-1a mRNA expression in heart, kidney, liver, lung, bone marrow, spleen, chondrocytes, cartilage, retina and muscle. Other workers have reported GLAST-1 expression in lung, spleen, skeletal muscle, testis [19], erythrocytes [20], mammary gland [21] and placenta-[22]. In addition NMDA receptors have been detected in cardiocytes [23], ileum [24], pancreas [25] and have been shown to be involved in pulmonary oedema [26]. These data also implicate a role for GLAST-1a in treatment of disorders of the tissues listed.

The inventors have appreciated that among other things, different levels of expression in different tissues along with variations in untranslated regions of the molecules across splice variants may allow tissue-specific targeting of therapeutic agents. For-example, the 3′ untranslated region of the GLAST-1 gene may well contain variations in it sequence according to the tissue it is found in. These variations may therefore lead to the differential expression of the splice variant making it tissue specific. This opens up the possibility of using these variations as tissue markers or for specifically targeting certain tissues.

The present inventors have detected the expression of a novel variant of GLAST-1 that excises exon 3 in rat bone, cartilage, retina and brain. Loss of exon 3 does not alter reading frame and results in the removal of 46 amino acid residues. This would considerably alter the structure of the protein that might be encoded by this transcript. Prediction of GLAST-1 protein structure suggests that there are 6 transmembrane (TM) domains in the N terminal (Slotboom, 1999). The first 3 TM domains are coded by exons 2,3 and 4 in GLAST-1 (FIG. 3a). However, GLAST-1a only possesses exons 1,2 and 4 (FIG. 3b). The inventors predict GLAST-1a protein will lose the first extracellular domain and a portion of the first and second TM domains such that the first and second hydrophobic regions fuse to generate a single TM domain (FIG. 4).

The assembly of transmembrane proteins is not fully understood. The most simple eukaryotic model is sequential start stop transfer where hydrophobic sequences insert into the plasma membrane one after the other in an orientation governed by the most N-terminal sequence. This model was questioned as the only mechanism of membrane protein assembly by (Gafvelin, 1997), who demonstrated that the presence or absence of positively charged residues in the most N-terminal non-hydrophobic region orientate it cytoplasmically or luminal respectively. Interestingly, unlike the prokaryotic system, charged residues on subsequent non-hydrophobic regions have less of an influence on orientation, with highly charged loops capable of being translocated into the ER lumen (Gafvelin et al 1997). A recent review (Slotboom, 1999) of structure and function of known Na⁺ dependent EAA transporters predicts that the N-terminal of GLAST-1, which has five arginyl and eight lysyl residues, would be cytoplasmic. The inventors therefore predict that if GLAST-1a is translated then its N terminal would be cytoplasmic. As subsequent non-hydrophobic regions have less influence in eukaryotes the loss of exon 3, converting 3 hydrophobic regions into 2, could have the effect of flipping the C-terminal of the protein and pore orientation (FIG. 4b). If this were the case then the second large extracellular domain of GLAST-1, that is glycosylated at asparagines 206 and 216 (Conradt 1995), would become cytoplasmic and therefore not presented for glycosylation within the ER lumen. Assuming no further post-translational modification, the unglycosylated GLAST-1a would have a molecular weight of 54.4 kDa. The inventors have detected an approximately 55 kDa immunoreactive protein-on western blots of brain protein using an anti-GLAST antibody (FIG. 6). They believe that this represents unglycosylated GLAST-1a, supporting the reversed orientation theory.

There is evidence that GLAST transports EAA in both directions across the cell membrane (Dr Paul Chapman personal communication and Rossi et al, 2000 Billups 1998) and the novel splice variant described herein encodes an ideal candidate protein for reversal of glutamate uptake.

The inventors have confirmed that an mRNA molecule that possesses the open reading frame for GLAST-1 is being expressed in bone. This work along with the discovery of functional glutamate receptors on bone cells (Lakatic-Ljubokevic 1999, Gu 2000) suggests that glutamate signalling is playing a key role in bone cell signalling.

As discussed above, the inventors have also discovered a splice variant of the GLAST-1 gene that is expressed in rat bone, brain, cartilage, retina and SaOS-2 osteoblasts). This molecule does not contain exon 3 of GLAST-1 but otherwise possesses the rest of the open reading frame. Loss of exon three potentially flips the C-terminal into an opposite orientation to that of GLAST-1 and may provide some valuable information in the study of transmembrane protein formation. The inventors believe that GLAST-1a works in the opposite orientation to GLAST-1, pumping glutamate from inside to outside the cell. Thus, the understanding of how these molecules function provides potential targets for therapeutic treatments to diseases that may result from a breakdown in glutamate signalling, or be influenced by it.

Therefore, at its most general, the present invention provides materials and methods relating to the splice variant GLAST-1a including the amino acid and nucleic acid sequence; materials and methods relating to the detection in vivo or in vitro of the GLAST-1a; and materials and methods relating to the modulation of EAA signalling.

Thus, in a first aspect of the present invention, there is provided a nucleic acid molecule encoding splice variant GLAST-1a. Preferably, the nucleic acid molecule comprises the nucleic acid sequence as provided in FIG. 5a. Further, there is provided a nucleic acid molecule which has a nucleic acid sequence encoding a GLAST-1a polypeptide including the amino acid sequence set out in FIG. 5b.

In all cases, the nucleic acid sequence may be a mutant, variant, derivative or allele of the nucleic acid sequence set out in FIG. 5a, or, the nucleic acid molecule may encode a polypeptide which is a mutant, variant, derivative or allele of the nucleic acid sequence set out in FIG. 5b.

The coding sequence may be that shown in FIG. 5a or it may be a mutant, variant, derivative or allele of these sequences. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in FIG. 5a yet encode a polypeptide with the same amino acid sequence. The amino acid sequence of the complete GLAST-1a polypeptide shown in FIG. 5b consists of 497 residues.

On the other hand, the encoded polypeptide may comprise an amino acid sequence which differs by one or more amino acid residues from the amino acid sequence shown in FIG. 5a. Nucleic acid encoding a polypeptide which is an amino acid sequence mutant, variant, derivative or allele of the sequence shown in FIG. 5b is further provided by the present invention. Such polypeptides are discussed below. Nucleic acid encoding such a polypeptide may show greater than about 60% homology with the coding sequence shown in FIG. 5a, greater than about 70% homology, greater than about 80% homology, greater than about 90% homology or greater than about 95% homology.

Generally, nucleic acid of the present invention is provided as an isolate, in isolated and/or purified form, or free or substantially free of material with which it is naturally associated. The nucleic acid of the splice variant will usually be in the form of RNA or cDNA derived from the mRNA. Where nucleic acid according to the invention includes RNA, reference to the sequence shown should be construed as reference to the RNA equivalent, with U substituted for T.

Nucleic acid sequences encoding all or part of the GLAST-1a variant can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992). These techniques include (i) the use of the polymerase chain reaction (PCR) to amplify samples of such nucleic acid, (ii) chemical synthesis, or (iii) preparing cDNA sequences. Modifications to the GLAST-1a sequences can be made, e.g. using site directed mutagenesis, to lead to the expression of modified GLAST-1a polypeptide or to take account of codon preference in the host cells used to express the nucleic acid.

In order to obtain expression of the GLAST-1a nucleic acid sequences, the sequences can be incorporated in a vector having control sequences operably linked to the GLAST-1a nucleic acid to control its expression. The vectors may include other sequences such as promoters, enhancers or repressors to drive and control the expression of the inserted nucleic acid, nucleic acid sequences so that the GLAST-1a polypeptide is produced as a fusion and/or nucleic acid encoding secretion signals so that the polypeptide produced in the host cell is secreted from the cell. The GLAST-1a polypeptide can then be obtained by transforming the vectors into host cells in which the vector is functional, culturing the host cells so that the GLAST-1a polypeptide is produced and recovering the GLAST-1a polypeptide from the host cells or the surrounding medium. Prokaryotic and eukaryotic cells are used for this purpose in the art, including strains of E. coli, yeast, and eukaryotic cells such as COS or CHO cells. The choice of host cell can be used to control the properties of the GLAST-1a polypeptide expressed in those cells, e.g. controlling where the polypeptide is deposited in the host cells or affecting properties such as its glycosylation.

In accordance with the above, recombinant expression constructs may be provided for the expression of GLAST-1a sense or antisense sequences in prokaryotic and eukaryotic systems. These constructs may be used to transfect mammalian cells in order to express GLAST-1a mRNA and protein. These may then be used for investigation of novel transporter structure, function and to assay compounds that effect EAA uptake.

PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises steps of denaturation of template nucleic acid (if double-stranded), annealing of primer to target, and polymerisation. The nucleic acid probed or used as template in the amplification reaction may be genomic DNA, mitochondrial DNA, cDNA or RNA. PCR can be used to amplify specific sequences from specific RNA sequences and cDNA transcribed from mRNA, bacteriophage or plasmid sequences. The GLAST-1a nucleic acid sequences provided herein readily allow the skilled person to design PCR primers. References for the general use of PCR techniques include Mullis et al, Cold Spring Harbor Symp. Quant. Biol., 51:263, (1987), Ehrlich (ed), PCR technology, Stockton Press, NY, 1989, Ehrlich et al, Science, 252:1643-1650, (1991), “PCR protocols; A Guide to Methods and Applications”, Eds. Innis et al, Academic Press, New York, (1990).

Also included within the scope of the invention are antisense oligonucleotide sequences based on the GLAST-1a nucleic acid sequences described herein. Antisense oligonucleotides or pDNA may be designed to hybridise to the promoter or regulatory elements of GLAST-1a or the complementary sequence of nucleic acid, pre-mRNA or mature mRNA, interfering with the production of polypeptide encoded by a given DNA sequence (e.g. either native GLAST-1a polypeptide or a mutant form thereof), so that its expression is reduced or prevented altogether. The construction of antisense sequences and their use is described in Peyman and Ulman, Chemical Reviews, 90:543-584, (1990), Crooke, Ann. Rev. Pharmacol. Toxicol., 32:329-376, (1992), and Zamecnik and Stephenson, P.N.A.S, 75:280-284, (1974). For example, antisense oligonucleotides may be designed to hybridize to mRNA encoding GLAST-1a thereby preventing translation of said mRNA and the production of the GLAST-1a polypeptide. Alternatively, nucleic acid probes may be designed to hybridize to 3′ untranslated regions of the GLAST-1 gene which contain variations in their sequence resulting in the production of the GLAST-1a splice variant.

The nucleic acid sequences provided in FIG. 5a are useful for identifying nucleic acid of interest (and which may be according to the present invention) in a test sample. The present invention provides a method of obtaining nucleic acid of interest, the method including hybridisation of a probe having the sequence derived from the sequence shown in FIG. 5a or a complementary sequence, to target nucleic acid.

Hybridisation is generally followed by identification of successful hybridisation and isolation of nucleic acid which has hybridised to the probe, which may involve one or more steps of PCR.

In accordance with the present invention, nucleic acids having the appropriate level of sequence homology with the splice variant GLAST-1a as shown in FIG. 5a may be identified by using hybridization and washing conditions of appropriate stringency. For example, hybridizations may be performed, according to the method of Sambrook et al., (22) using a hybridization solution comprising: 5×SSC, 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C. +16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

As the nucleic acid in accordance with the present invention is a splice variant, it will be present in cells as mRNA. The mRNA encoding GLAST-1a will differ from that encoding GLAST-1 by missing the sequence of exon 3. Thus, the two sequences will differ in length by approximately 138 nucleotides. This difference will serve to distinguish between the mRNA encoding GLAST-1a from other transcripts encoding the GLAST-1 protein.

Oligonucleotide probes or primers, as well as the full length GLAST-1a sequence (and mutants, alleles, variants and derivatives) are also useful in screening a test sample for the presence or absence of the splice variant GLAST-1a. Nucleic acid primers may be designed so as to amplify nucleic acid spanning exon 3 of GLAST-1. The amplified nucleic acid sequences may then be separated according to size on an appropriate electrophoresis gel. Those sequences amplified from GLAST-1 transcripts will be larger by approximately 140 nucleotides than those amplified from GLAST-1a transcripts. Thus, the gel will identify an additional band of amplified nucleic acid not seen on gels containing GLAST-1 transcripts. Primers may also be designed to the exon 2 to 4 boundary of GLAST-1a for specific amplification of GLAST-1a. These methods would of identify cells or tissues which express GLAST-1a.

An oligonucleotide probe designed from the sequence set out in FIG. 5a (i.e. containing contiguous sequence flanking exon 3 but not containing exon 3) may be used to specifically identify GLAST-1a transcripts in a sample. Such an oligonucleotide sequence should not specifically bind to GLAST-1 transcripts as they will contain the exon 3 nucleic acid sequence (approximately 140 nucleotides between the two flanking sequences).

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RNAase cleavage and allele specific oligonucleotide probing.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

An oligonucleotide primer for use in nucleic acid amplification may have about 10 or fewer codons (e.g. 6, 7 or 8), i.e. be about 30 or fewer nucleotides in length (e.g. 18, 21 or 24). Generally specific primers are upwards of 14 nucleotides in length, but not more than 18-20. Those skilled in the art are well versed in the design of primers for use processes such as PCR.

A further aspect of the present invention provides an oligonucleotide or polynucleotide fragment of the nucleotide sequence shown in FIG. 5a or a complementary sequence, in particular for use in a method of obtaining and/or screening nucleic acid. The sequences referred to above may be modified by addition, substitution, insertion or deletion of one or more nucleotides, but preferably without abolition of ability to hybridise selectively with nucleic acid with the sequence shown in FIG. 5a, that is wherein the degree of homology of the oligonucleotide or polynucleotide with one of the sequences given is sufficiently high.

In some preferred embodiments, oligonucleotides according to the present invention that are fragments of any of the sequence shown in FIG. 5a, are at least about 10 nucleotides in length, more preferably at least about 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of a sequence indicative of the presence of GLAST-1a.

As mentioned above the present invention also provides a GLAST-1a polypeptide having a sequence as shown in FIG. 5b or a fragment thereof.

A convenient way of producing a polypeptide according to the present invention is to express nucleic acid encoding it, by use of the nucleic acid in an expression system. The use of expression system has reached an advanced degree of sophistication today.

Accordingly, the present invention also encompasses a method of making a polypeptide (as disclosed), the method including expression from nucleic acid encoding the polypeptide (generally nucleic acid according to the invention). This may conveniently be achieved by growing a host cell in culture, containing such a vector, under appropriate conditions which cause or allow expression of the polypeptide. Polypeptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Thus, the present invention also provides a host cell containing nucleic acid as disclosed herein. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

Further, the present invention also provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers. Introduction of nucleic acid may take place in vivo by way of gene therapy.

As mentioned above, the present invention provides a polypeptide which has the amino acid sequence shown in FIG. 5b, which may be in isolated and/or purified form, free or substantially free of material with which it is naturally associated, such as other polypeptides or such as human polypeptides other than GLAST-1a polypeptide or (for example if produced by expression in a prokaryotic cell) lacking in native glycosylation, e.g. unglycosylated.

Polypeptides which are amino acid sequence variants, alleles, derivatives or mutants are also provided by the present invention. A polypeptide which is a variant, allele, derivative or mutant may have an amino acid sequence which differs from that given in FIG. 5b by one or more of addition, substitution, deletion and insertion of one or more amino acids. Preferred such polypeptides have GLAST-1a function, that is to say have one or more of the following properties: immunological cross-reactivity with an antibody reactive the polypeptide for which the sequence is given in FIG. 5b; sharing an epitope with the polypeptide for which the amino acid sequence is shown in FIG. 5b (as determined for example by immunological cross-reactivity between the two polypeptides.

A polypeptide which is an amino acid sequence variant, allele, derivative or mutant of the amino acid sequence shown in FIG. 5b may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. The sequence may share greater than about 60% similarity, greater than about 70% similarity, greater than about 80% similarity or greater than about 90% similarity with the amino acid sequence shown in FIG. 5b. Particular amino acid sequence variants may differ from that shown in FIG. 5b by insertion, addition, substitution or deletion of 1 amino acid, 2, 3, 4, 5-10, 10-20 20-30, 30-50, 50-100, 100-150, or more than 150 amino acids.

A polypeptide, peptide fragment, allele, mutant or variant according to the present invention may be used as an immunogen or otherwise in obtaining specific antibodies. Antibodies are useful in purification and other manipulation of polypeptides and peptides, diagnostic screening and therapeutic contexts.

A polypeptide according to the present invention may be used in screening for molecules which affect or modulate its activity or function. Such molecules may be useful in a therapeutic (possibly including prophylactic) context.

A number of methods are known in the art for analysing biological samples from individuals to determine whether the individual expresses the splice variant or expresses it at different levels or in different tissues. The purpose of such analysis may be used for diagnosis or prognosis, and serve to detect the presence of an existing disease, to help identify the type of disease, to assist a physician in determining the severity or likely course of the disease and/or to optimise treatment of it. Alternatively, the methods can be used to detect transcripts of splice variants that are statistically associated with a susceptibility to certain diseases states in the future, identifying individuals who would benefit from regular screening to provide early diagnosis of the disease state.

Broadly, the methods divide into those screening for the presence of GLAST-1a nucleic acid sequences (mRNA or variations in the GLAST-1 genomic DNA that may lead to the splice variant being transcribed, or to the control elements of the GLAST-1 gene, e.g. the 3′ untranslated region which may lead to the splice variant being transcribed) and those that rely on detecting the presence or absence of the GLAST-1a polypeptide. The methods make use of biological samples from individuals that are suspected of containing the nucleic acid sequences or polypeptide. Examples of biological samples include blood, plasma, serum, tissue samples, tumour samples, saliva and urine.

Exemplary approaches for detecting GLAST-1a nucleic acid or polypeptides include:

(a) comparing the sequence of nucleic acid in the sample with the GLAST-1a nucleic acid sequence to determine whether the sample from the patient contains the splice variant GLAST-1a; or,

(b) determining the presence in a sample from a patient of the polypeptide encoded by the GLAST-1a transcript; or,

(c) using a specific binding member capable of binding to a GLAST-1a mRNA nucleic acid sequence, the specific binding member comprising nucleic acid hybridisable with the GLAST-1a sequence, or substances comprising an antibody domain with specificity for the GLAST-1a nucleic acid sequence or the polypeptide encoded by it, the specific binding member being labelled so that binding of the specific binding member to its binding partner is detectable; or,

(d) using PCR involving one or more primers derived from sequence spanning exon 3 of GLAST-1 or derived from exon 2 to 4 junction of GLAST-1a as shown in FIG. 2b to screen for transcripts of the splice variant GLAST-1a in a sample from a patient.

A “specific binding pair” comprises a specific binding member (sbm) and a binding partner (bp) which have a particular specificity for each other and which in normal conditions bind to each other in preference to other molecules. Examples of specific binding pairs are antigens and antibodies, molecules and receptors and complementary nucleotide sequences. The skilled person will be able to think of many other examples and they do not need to be listed here. Further, the term “specific binding pair” is also applicable where either or both of the specific binding member and the binding partner, comprise a part of a larger molecule. In embodiments in which the specific binding pair are nucleic acid sequences, they will be of a length to hybridise to each other under the conditions of the assay, preferably greater than 10 nucleotides long, more preferably greater than 15 or 20 nucleotides long.

In most embodiments for screening for GLAST-1a splice variant, the GLAST-1a nucleic acid in the sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art.

Thus, in a second aspect of the present invention, there is provided a method of determining the presence or absence of the splice variant GLAST-1a in a biological test sample using a nucleic acid probe having all or a portion of the nucleic acid sequence shown in FIG. 2b or a complementary sequence thereof, the method comprising contacting the probe and the test sample under hybridising conditions and observing whether hybridization takes place.

In a fourth aspect of the present invention, there is provided a method of determining the presence or absence of the splice variant GLAST-1a in a biological sample using a first and a second oligonucleotide primer designed from the sequence provided in FIG. 2b such that said first and second oligonucleotide primers hybridise to sequence flanking exon 3 of GLAST-1, contacting said oligonucleotide primers with the biological sample under conditions suitable for annealing, elongation and denaturation in accordance with PCR; and determining the present or absence of an amplified nucleic acid sequence corresponding to the presence of exon 3.

In a third aspect of the present invention, there is provided a method of determining the presence or absence of a GLAST-1a polypeptide in a test biological sample, using a specific binding member capable of specifically binding to the GLAST-1a polypeptide, said method comprising the step of contacting the specific binding member and the test sample under binding conditions and observing whether binding takes place. Preferably, the specific binding member is an antibody binding domain. More preferably, the antibody binding domain is labelled so that specific binding may be observed.

Antibodies may be raised by a GLAST-1a polypeptide according to the present invention. Thus, a further important use of the GLAST-1a polypeptide is in raising antibodies that have the property of specifically binding to the GLAST-1a polypeptide, or fragments or active portions thereof. Preferably as polypeptide sequence corresponding to the exon 2 to 4 junction is used to raise such antibodies.

The production of monoclonal antibodies is well established in the art. Monoclonal antibodies can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-239400. A hybridoma producing a monoclonal antibody may be subject to genetic mutation or other changes, which may or may not alter the binding specificity of antibodies produced.

The provision of the novel GLAST-1a polypeptide enables for the first time the production of antibodies able to bind it specifically. Accordingly, a further aspect of the present invention provides an antibody able to bind specifically to the polypeptide whose sequence is given in FIG. 5b. Such an antibody may be specific in the sense of being able to distinguish between the polypeptide it is able to bind and GLAST-1 polypeptides for which it has no or substantially no binding affinity (e.g. a binding affinity of about 1000× worse). Specific antibodies bind an epitope on the molecule which is either not present or is not accessible on other molecules.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal.

As an alternative or supplement to immunising a mammal with a peptide, an antibody specific for a protein may-be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanised antibodies in which CDRs from a non-human source are grafted onto human framework regions, typically with the alteration of some of the framework amino acid residues, to provide antibodies which are less immunogenic than the parent non-human antibodies, are also included within the present invention

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Hybridomas capable-of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The mode of determining binding is not a feature of the present invention and those skilled in the art are able to choose a suitable mode according to their preference and general knowledge.

Antibodies according to the present invention may be used in screening for the presence of a GLAST-1a polypeptide, for example in a test sample containing cells or cell lysate as discussed, and may be used in purifying and/or isolating a polypeptide according to the present invention, for instance following production of the polypeptide by expression from encoding nucleic acid therefor. Antibodies may modulate the activity of the polypeptide to which they bind and so, if that polypeptide has a deleterious effect in an individual, may be useful in a therapeutic context (which may include prophylaxis).

Further, an antibody which can specifically bind GLAST-1a may be used in a screening method to test the effects of pharmaceutical compounds on form example GLAST mediated signalling. By using such an antibody, GLAST-1a may effectively be blocked and it can then be determined whether the pharmaceutical compound works through GLAST 1a or not. It is well known that pharmaceutical research leading to the identification of a new drug may involve the screening of very large numbers of candidate compounds, both before and even after a lead compound has been found. This is one factor that makes pharmaceutical research very expensive and time-consuming. Means for assisting in the screening process can therefore have considerable commercial importance.

An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

Nucleic acids, polypeptides and/or antibodies according to the present invention may form part of a pharmaceutical composition for the treatment of diseases that result from, or are affected by EAA levels, e.g. in the CNS, bone, eye, joints or skin. For example, pharmaceutical compositions may be used to modulate EAA signalling to control diseases of the CNS. Further, pharmaceutical compositions may be used to modulate bone turnover in diseases of bone. Other pharmaceutical compositions may be used to treat other diseases e.g. of the CNS, eye, joints or skin.

Thus, a further aspect of the present invention provides the use of nucleic acids, polypeptides or antibodies as described above in the preparation of medicaments to treat diseases, specifically diseases associated with GLAST mediated signalling, e.g. EAA signalling. Such diseases may be of the CNS, bone, eye, joints or skin. For example, an antisense nucleic acid molecule of GLAST-1a may be capable of hybridising to the complementary sequence of the GLAST-1a nucleic acid, pre-mRNA or mature mRNA so that expression of the GLAST-1a nucleic acid is reduced or prevented. This use may be a form of gene therapy.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE DRAWINGS

FIG. 1. RT-PCR using primers to GLAST-1 exons 1 to 10 revealed expression of expected 2201 bp product in bone.

FIG. 2a. EcoR1 digest of cloned products from exon 1-10 PCR revealed 2 different sized inserts.

FIG. 2b. BLAST2 comparison of the bone cDNA clones, derived from exon 1-10 PCR, illustrating the absence of exon 3 in GLAST-1a (Seq. A). All other sequence comparisons, to date, are identical between two sequences and the GLAST-1 sequence published by Tanaka et al 1993 (Accession number S59158).

FIG. 2c. RT-PCR using GLAST-1a specific primers revealed the expected 210bp product, rat tibia (1) rat brain (2) and water control (3).

FIG. 3. Hydrophobicity plots of the amino acids to exon 4 of a) GLAST-1 and b) GLAST-1a. FIG. 3a shows 3 TM domains in GLAST-1 and FIG. 3b reveals 2TM domains in GLAST-1a.

FIG. 4. Topological model of a) GLAST-1 (Seal 2000) and our hypothetical model of b) GLAST-1a. The loss of exon 3 transforms the first two transmembrane domains into one, which we predict will flip the C-terminal and reverse glutamate (E) transport. Note extracellular asparagine residues N²⁰⁶ and N²¹⁶ become intracellular in GLAST-1a.

FIG. 5(a). The nucleotide sequence for the bone derived cDNA of GLAST-1a.

FIG. 5(b). The predicted amino acid sequence of the nucleotide sequence given in FIG. 5a.

FIG. 6. Western blot analysis using anti-GLAST antibody. Lanes 1 & 2=cerebellum crude and membrane enriched fractions respectively. Lanes 3 & 4=bone crude and membrane enriched fractions respectively. Band sizes shown in kDa±SD derived from 3 independent determinations. Bands marked with * are due to non-specific binding of secondary antibody.

### DETAILED DESCRIPTION

Tissue Preparation and RNA Extraction

Rat tibia were dissected from wistar rats and epiphyses removed. Diaphyses were placed into 1.5 ml centrifuge tubes and marrow flushed from cavity by centrifugation at 1000 rpm for 30 seconds. Bone was then snap frozen in liquid nitrogen and dismembrated (2000 rpm for 3 minutes at approximately 120° C.) rat tibia using 1 ml TRIZOL® reagent (GIBCO, BRL). Total RNA extracted from following manufacturers instructions. RNA was precipitated with 0.5% v/v isopropanol and 0.05% Tack. resin (Biogenesis). RNA was also extracted from 100 mg of whole rat brain as above. Contaminating gnomic DNA was removed from all RNAs using DNase (Promega) following manufacturers instructions. RNA concentration was estimated using a spectrophotometer (Pharmacia, Biotech) measuring wavelength at 260 nm and 280 nm, where 1 unit of absorbance at 260 is equivalent to 40 μg/ml of RNA, the 260/280 absorbance ratio was used to determine purity of RNA and accuracy of reading.

RT-PCR and Cloning of Amplicons

2.5 μg of Oligo dT₍₁₅₎ primed RNA was reverse transcribed using Superscript™ II (GIBCO, BRL) according to manufacturers instructions. PCR primers designed to sequences in exons 1 (down stream TCCACCAGTCACAGAATCAGA) and 10 (upstream GAGTCAGAAGAAAGGGCAAAC) of the published GLAST-I sequence (genbank accession number S59158) were used to amplify the GLAST-1 cDNA. PCR was performed using Advantage DNA polymerase (Clontech) for 40 cycles at 95° C. for 1 minute, 63° C. for 1 minute and 72° C. for 2½ minutes. Amplicons were incubated at 95° C. for 20 minutes to inactivate proof reading enzyme and adenosine overhangs added by adding 5U of Taq polymerase (AGS gold: Hybaid) and incubating for 20 minutes at 72° C. Amplicons were then cloned into pCR®-XL-TOPO (Invitrogen) following manufacturers instructions. Transformed plasmids were purified (Wizard®-SV Plus miniprep kit Promega) and inserts sequenced using M13 vector primers and forward and reverse sequencing primers designed to published sequence (accession No. S59158).

Confirmation of GLAST-1a Splice Variant

Primers were designed to specifically amplify the GLAST-1a splice variant. The forward primer (CAGCGCTGTCA TTGTGGGAATGGC) was designed to prime across the exon 2-4 boundary and the reverse primer was designed to the 3′ end of exon 4 (AGGAAGGCATCTGCGGCAGTCACC). This reaction was performed using taq polymerase (AGS gold: Hybaid) for 40 cycles at 95° C. for 1 minute, 58° C. for 1 minute and 72° C. for 2 minutes.

Structural Analysis

Hydrophobicity plots were performed using TM pred at web address: http://www.embnet.org/software/TMPRED_form.html

GLAST-1 cDNA from bone

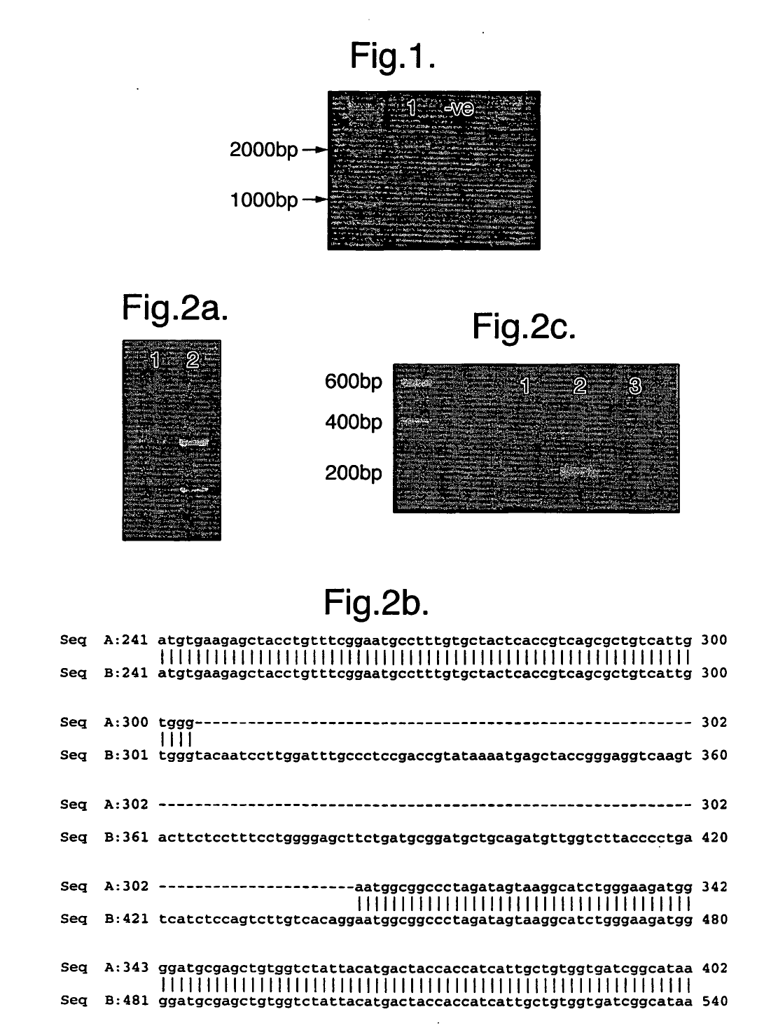

RT-PCR of bone RNA using primers to exons 1 and 10 of the published GLAST-1 sequence (Storck, 1992) yielded an amplicon of the expected 2201 bp for this molecule (FIG. 1). Sequence analysis confirmed that this bone-derived PCR product contained the complete open reading frame of the GLAST-1 mRNA previously thought to be exclusively expressed in the central nervous system of both rats and humans (Tanaka, 1993).

A Splice Variant that Excises exon 3

Eco RI restriction digest of cloned exon 1-10 PCR products yielded two different sized inserts (FIG. 2a). Comparison of DNA sequence data revealed a novel variant of GLAST-1 mRNA that does not possess exon 3 (FIG. 2b). This variant has been called GLAST-1a. RT-PCR, using an upstream primer to the exon 2-4 boundary and a downstream primer to exon 4 to specifically amplify GLAST-1a, demonstrated that it is expressed in brain as well as bone (FIG. 2c).

Transmembrane Modelling

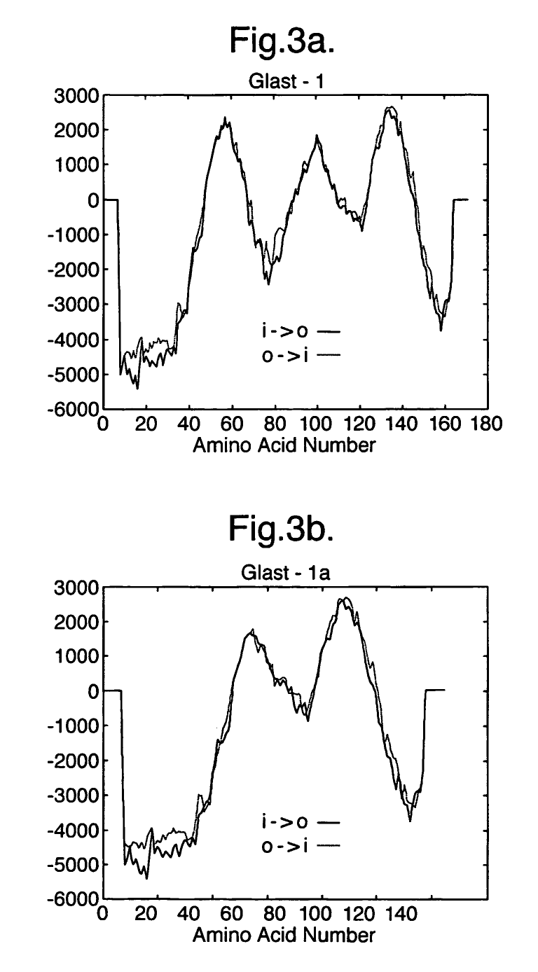

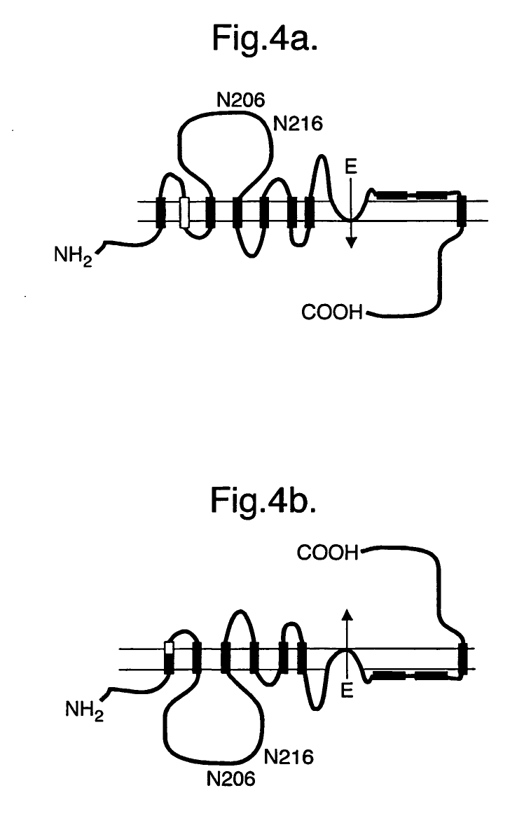

Transmembrane (TM) prediction of the first four exons of GLAST-1 reveals that it has three hydrophobic regions that may correspond to TM domains (FIG. 3a). Interestingly TM prediction of the hypothetical protein without exon three reveals that there are only two hydrophobic regions which would correspond to just two transmembrane domains (FIG. 3b). Loss of exon three alters the N-terminal region from three potential TM domains (GLAST-1) to two (GLAST-1a) which may result in reorientating the C-terminal (FIG. 4).

Western Blot Analysis using Anti-GLAST Antibody

Immunoblot analysis was used to confirm the presence of GLAST-1 protein expression in long bones and to identify GLAST isoforms present in rat cerebellum. Lyophilized fractions were dissolved in sample buffer (8M urea, 2M thiourea, 5% (w/v) SDS, 25mM Tris-HCI (pH 7.5), 1% (w/v) bromophenol blue and 5% (v/v) β-mercaptoethanol) to a final concentration of 10 mg/ml and incubated at 60° C. for 15 minutes. 50 μg of-each extract were resolved on 7.5% or 10% SDS-polyacrylamide gels and subsequently transferred to polyvinyldifluoride membrane (Immobilon-PVDF, Millipore). 5 μl of prestained SDS-PAGE protein standards (Bio-Rad Laboratories) were also resolved on each gel and the mobilities of these standards (molecular weights 28.5 KDa to 113 KDa) were used to determine molecular weight of GLAST isoforms.

Non-specific binding sites on the membrane were blocked by incubating in 1% (w/v) skimmed milk powder in TBS (0.05M Tris-HS1, pH 8.0, 0.15M NaCl) for 30 minutes. Membranes were incubated sequentially with an antibody preparation that recognises amino acids 24-40 of the rat GLAST-1 protein (kindly provided by Wilhelm Stoffel, University of Cologne [5]), diluted 1:1000 in TBS containing 0.2% (v/v) Tween 20 (TBS-Tween) and horse-radish peroxidase conjugated anti rabbit IgG diluted 1:1000 with TBS-tween. An additional blot was incubated without primary antibody to control for non-specific binding of secondary antibody. Membranes were washed extensively in between incubations with TBS-Tween. Specific binding of the anti GLAST-1 antibody was detected by enhanced chemiluminescence on Hyperfilm-ECL (Amersham, UK).

References

Birch, M. A., A. Patton J. et al (1997) J. Bone Miner. Res. 12: S411

Gafvelin, G. M. Sakaguch et al (1997).J. Biol. Chem., 272 (10): 6119-6127.

Mason D. J., Suva L. et al (1997) Bone 20 (3) 199-205.

Seal R. P., S. Amara G. (1998) Neuron 21: 1487-1498.

Slotboom, D. J. W. Konings, N. et al (1999). Microbiological and Molecular Biology Reviews: 293-307

Storck, T. S. Shculte et al (1992). Proc. Natl. Acad. Sci USA 89: 10955-10959

Tanaka K. (1993) Neurosci. Let. 159: 183-186.

Wahle, S. and W. Stoffel (1996) The journal of cell Biology 135(6): 1867-1877.

Obrenovitch, T. P. (1996) Origins of glutamate release in ischaemia: Acta Neurochir Suppl 66:50-55.

Conradt, Marcus et al (1995) Localisation of N-glycosylation sites and functional role of the carbohydrate units of GLAST-1, a cloned rat brain L-glutamate/L-aspartate transporter. European Journal of: Biochemistry 229: 682-687.

Billups, B. et al (1998) Patch-clamp, ion-sensing and glutamate-sensing techniques to study glutamate transport in isolated retinal glial cells. Methods in Enzymology 296: 617-632.

Seal, R, P. et al. A Model for the Topology of Excitatory Amino Acid Transporters Determined by the Extracellular Accessibility of Substituted Cysteines. Neuron 25: 695-706.

[1] Scott, H. L. et al. (1995) J Neurochem 64, 2193-202.

[2] Choi, D. W. et al. (1988) Neuron 1, 623-34.

[3] Huggett, J. et al. (2000) Febs Letter in press.

[4] Naskar, R. et al. (2000) Invest Ophthalmol Vis Sci 41, 1940-4.

[5] Vorwerk, C. K. et al. (2000) Invest Ophthalmol Vis Sci 41, 3615-21.

[6] Gu, Y. and Publicover, S. (2000) Journal of Biological Chemistry [pub ahead of print] 2000.

[7] Laketic-Ljubojevic, I. et al. (1999) Bone 25, 631-7.

[8] Chenu, C. et al. (1998) Bone 22, 295-299.

[9] Genever, P. G. et al (1998) Bone 23, S414.

[10] Genever, P. G. et al. (1999) Blood 93, 2876-83.

[11] Patton, A. J. et al. (1998) Bone 22, 645-649.

[12] Skerry (2000) in: BSMB Meeting on Cell-cell/cell-matrix interactions September 11th , University of Newcastle upon Tyne.

[13] Schirrmacher, K. et al. (1998) Calcif Tissue Int 63, 134-9.

[14] McNearney, T. et al. (2000) J Rheumatol 27, 739-45.

[15] Lawand, N. B et al. (2000) Pain 86, 69-74.

[16] Westlund, K. N. (1992) Brain Res Rev 17, 15-27.

[17] Genever P. G. et al. (1999) J Invest Dermatol 112, 337-42.

[18] Albina, J. E. et al. (1993) J Surg Res 55, 97-102.

[19] Tanaka, K. (1993) Neuroscience Letters 159, 183-186.

[20] Sato, K. et al. (2000) The Journal of Biological Chemistry 275, 6620-6627.

[21] Martinez-Lopez I. et al. (1998) Molecular Membrane Biology 15, 237-242.

[22] Matthews J. et al. (1998) Am J Physiol 274, C603-14.

[23] Morhenn, V. B. et al. (1994) Eur J Pharmacol 268, 409-14.

[24] Shannon, H. E et al. (1989) J Pharmacol Exp Ther 251, 518-23.

[25] Weaver, C. D. et al. (1996) J Biol Chem 271, 12977-84.

[26] Said, S. I. et al (1996) Proc Natl Acad Sci USA 93, 4688-92.

1. A nucleic acid molecule encoding a splice variant of GLAST-1, said splice variant being deficient of exon 3.

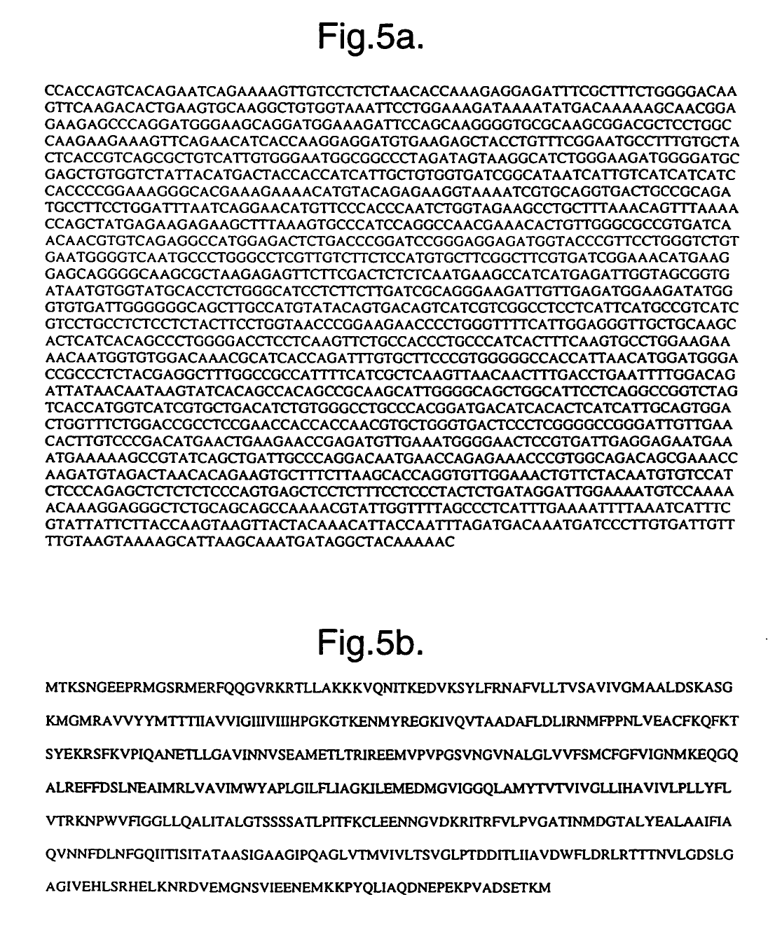

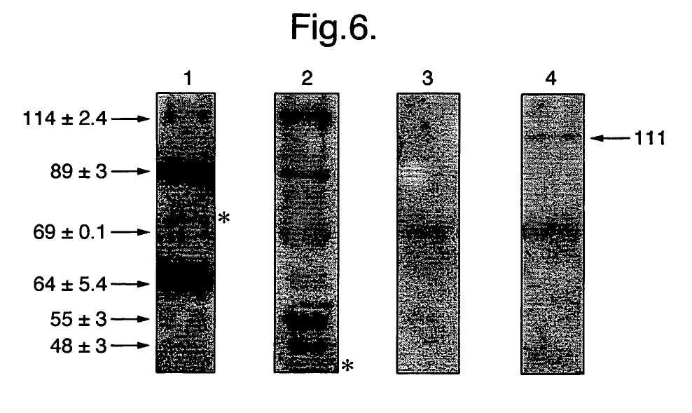

2. A nucleic acid molecule according to claim 1 having at least 80% homology with the nucleic acid sequence as shown in FIG. 5a.

3. A nucleic acid molecule according to claim 1 or claim 2 comprising the nucleic acid sequence as shown in FIG. 5a.

4. A nucleic acid molecule having a nucleic acid sequence encoding a GLAST-1a polypeptide, said polypeptide including an amino acid sequence having at least 80% homology with the amino acid sequence of FIG. 5b.

5. A nucleic acid molecule having a nucleic acid sequence encoding a GLAST-1a polypeptide including the amino acid sequence set out in FIG. 5b.

6. A replicable vector comprising a nucleic acid molecule according to any one of the preceding claims.

7. A host cell transformed with a nucleic acid molecule according to any one of claims 1 to 5, or a replicable vector according to claim 6.

8. A method of producing a GLAST-1a polypeptide comprising culturing the host cells of claim 7 so that the GLAST-1a polypeptide is produced.

9. The method of claim 8 comprising the further step of recovering the polypeptide produced.

10. A nucleic acid molecule according to any one of claims 1 to 5 further comprising a label.

11. A nucleic acid molecule according to any one of claims 1 to 5 for use in a method of medical treatment.

12. A polypeptide encoded by a nucleic acid molecule according to any one of claims 1 to 5.

13. A polypeptide including the amino acid sequence set out in FIG. 5b.

14. A polypeptide having 80% sequence homology to the GLAST-1a polypeptide including the amino acid sequence set out in FIG. 5b.

15. A substance which is a fragment or active portion or functional mimetic of a GLAST-1a polypeptide including the amino acid sequence of FIG. 5b.

16. A polypeptide according to any one of claims 12 to 14 or a substance according to claim 15 further comprising a label.

17. A polypeptide according to any one of claims 12 to 14, or a substance according to claim 15 for use in a method of medical treatment.

18. An antibody capable of specifically binding to a GLAST-1a polypeptide according to any one of claims 12 to 14.

19. An antibody according to claim 18 further comprising a label.

20. A pharmaceutical composition comprising a nucleic acid according to any one of claims 1 to 5, a polypeptide according to any one of claims 12 to 14, a substance according to claim 15, or an antibody according to claim 18.

21. A pharmaceutical composition according to claim 20 further comprising a pharmaceutically acceptable carrier.

22. A method of identifying a target nucleic acid molecule in a test sample using a nucleic acid probe having all or a portion of the sequence shown in FIG. 5a or a complementary sequence thereof, the method comprising contacting the probe and the test sample under hybridising conditions and observing whether hybridisation takes place.

23. Use of a nucleic acid molecule according to any one of claims 1 to 5, or a fragment thereof, in the preparation of a medicament for treating a condition associated with a change in glutamate signalling.

24. The use according to claim 23 wherein the nucleic acid molecule is an antisense oligonucleotide capable of hybridising to the complementary sequence of a GLAST-1a nucleic acid so that the expression of the GLAST-1a nucleic acid is reduced or prevented.

25. The use according to claim 24 wherein the nucleic acid molecule is an antisense oligonucleotide capable of hybridising to the complementary sequence of a GLAST-1a nucleic acid so that the expression of the GLAST-1a nucleic acid is increased.

26. The use of claim 24 or claim 25 wherein the use of the nucleic acid is in a method of gene therapy.

27. The use of a nucleic acid sequence as shown in FIG. 5a in the design of primers for use in the polymerase chain reaction.

28. The use of a nucleic acid sequence as shown in FIG. 5a in the design of a nucleic acid probe for detecting the presence of the GLAST-1a splice variant in a nucleic acid sample from a patient.

29. A method of detecting GLAST-1a nucleic acid splice variant or its encoded polypeptide comprising
(a) comparing the sequence of nucleic acid in the sample with the GLAST-1a nucleic acid sequence to determine whether the sample from the patient contains the splice variant GLAST-1a; or,
(b) determining the presence in a sample from a patient of the polypeptide encoded by the GLAST-1a transcript; or,
(c) using a specific binding member capable of binding to a GLAST-1a mRNA nucleic acid sequence, the specific binding member comprising nucleic acid hybridisable with the GLAST-1a sequence, or substances comprising an antibody domain with the specificity for the GLAST-1a nucleic acid sequence or the polypeptide encoded by it, the specific binding member being labelled so that binding of the specific binding member to its binding partner is detectable; or,
(d) using PCR involving one or more primers derived from sequence spanning exon 3 of GLAST-1 or derived from exon 2 to 4 junction of GLAST-1a as shown in FIG. 2b to screen for transcripts of the splice variant GLAST-1a in a sample from a patient.

30. A method of screening for substances which affect or modulate the activity of a GLAST-1a polypeptide according to any one of claims 12 to 14, the method comprising contacting one or more test substances with the GLAST-1a polypeptide in a reaction medium, testing the activity of the treated GLAST-1a polypeptide and comparing that activity with the activity of the GLAST-1a polypeptide in comparable reaction medium untreated with the test substance or substances.

31. A method of screening for substances which affect or modulate the activity of a GLAST-1a nucleic acid molecule according to any one of claims 1 to 5, the method comprising contacting one or more test substances with the GLAST-1a nucleic acid in a reaction medium, testing the activity of the treated GLAST-1a nucleic acid molecule and comparing that activity with the activity of the GLAST-1a polypeptide in comparable reaction medium untreated with the test substance or substances.

32. A method according to claim 31 wherein said, substances affect or modulate the expression of said GLAST-1a nucleic acid molecule.


SEQUENCE LISTING (8 sequences)

SEQ ID NO: 1 - DNA, 1929 bp, Rattus sp.
ccaccagtcacagaatcagaaaagttgtcctctctaacaccaaagaggagatttcgctttctggggacaagttcaagacactgaagtgcaaggctgtggtaaattcctggaaagataaaatatgacaaaaagcaacggagaagagcccaggatgggaagcaggatggaaagattccagcaaggggtgcgcaagcggacgctcctggccaagaagaaagttcagaacatcaccaaggaggatgtgaagagctacctgtttcggaatgcctttgtgctactcaccgtcagcgctgtcattgtgggaatggcggccctagatagtaaggcatctgggaagatggggatgcgagctgtggtctattacatgactaccaccatcattgctgtggtgatcggcataatcattgtcatcatcatccaccccggaaagggcacgaaagaaaacatgtacagagaaggtaaaatcgtgcaggtgactgccgcagatgccttcctggatttaatcaggaacatgttcccacccaatctggtagaagcctgctttaaacagtttaaaaccagctatgagaagagaagctttaaagtgcccatccaggccaacgaaacactgttgggcgccgtgatcaacaacgtgtcagaggccatggagactctgacccggatccgggaggagatggtacccgttcctgggtctgtgaatggggtcaatgccctgggcctcgttgtcttctccatgtgcttcggcttcgtgatcggaaacatgaaggagcaggggcaagcgctaagagagttcttcgactctctcaatgaagccatcatgagattggtagcggtgataatgtggtatgcacctctgggcatcctcttcttgatcgcagggaagattgttgagatggaagatatgggtgtgattggggggcagcttgccatgtatacagtgacagtcatcgtcggcctcctcattcatgccgtcatcgtcctgcctctcctctacttcctggtaacccggaagaacccctgggttttcattggagggttgctgcaagcactcatcacagccctggggacctcctcaagttctgccaccctgcccatcactttcaagtgcctggaagaaaacaatggtgtggacaaacgcatcaccagatttgtgcttcccgtgggggccaccattaacatggatgggaccgccctctacgaggctttggccgccattttcatcgctcaagttaacaactttgacctgaattttggacagattataacaataagtatcacagccacagccgcaagcattggggcagctggcattcctcaggccggtctagtcaccatggtcatcgtgctgacatctgtgggcctgcccacggatgacatcacactcatcattgcagtggactggtttctggaccgcctccgaaccaccaccaacgtgctgggtgactccctcggggccgggattgttgaacacttgtcccgacatgaactgaagaaccgagatgttgaaatggggaactccgtgattgaggagaatgaaatgaaaaagccgtatcagctgattgcccaggacaatgaaccagagaaacccgtggcagacagcgaaaccaagatgtagactaacacagaagtgctttcttaagcaccaggtgttggaaactgttctacaatgtgtccatctcccagagctctctctcccagtgagctcctctttcctccctactctgataggattggaaaatgtccaaaaacaaaggagggctctgcagcagccaaaacgtattggttttagccctcatttgaaaattttaaatcatttcgtattattcttaccaagtaagttactacaaacattaccaatttagatgacaaatgatcccttgtgattgttttgtaagtaaaagcattaagcaaatgataggctacaaaaac

SEQ ID NO: 2 - protein, 497 aa, Rattus sp.
MTKSNGEEPRMGSRMERFQQGVRKRTLLAKKKVQNITKEDVKSYLFRNAFVLLTVSAVIVGMAALDSKASGKMGMRAVVYYMTTTIIAVVIGIIIVIIIHPGKGTKENMYREGKIVQVTAADAFLDLIRNMFPPNLVEACFKQFKTSYEKRSFKVPIQANETLLGAVINNVSEAMETLTRIREEMVPVPGSVNGVNALGLVVFSMCFGFVIGNMKEQGQALREFFDSLNEAIMRLVAVIMWYAPLGILFLIAGKILEMEDMGVIGGQLAMYTVTVIVGLLIHAVIVLPLLYFLVTRKNPWVFIGGLLQALITALGTSSSSATLPITFKCLEENNGVDKRITRFVLPVGATINMDGTALYEALAAIFIAQVNNFDLNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNEPEKPVADSETKM

SEQ ID NO: 3 - DNA, 162 bp, Rattus sp.
atgtgaagagctacctgtttcggaatgcctttgtgctactcaccgtcagcgctgtcattgtgggaatggcggccctagatagtaaggcatctgggaagatggggatgcgagctgtggtctattacatgactaccaccatcattgctgtggtgatcggcataa

SEQ ID NO: 4 - DNA, 300 bp, Rattus sp.
atgtgaagagctacctgtttcggaatgcctttgtgctactcaccgtcagcgctgtcattgtgggtacaatccttggatttgccctccgaccgtataaaatgagctaccgggaggtcaagtacttctcctttcctggggagcttctgatgcggatgctgcagatgttggtcttacccctgatcatctccagtcttgtcacaggaatggcggccctagatagtaaggcatctgggaagatggggatgcgagctgtggtctattacatgactaccaccatcattgctgtggtgatcggcataa

SEQ ID NO: 5 - DNA, 21 bp, Artificial Sequence
  Description of Artificial Sequence Primer
tccaccagtcacagaatcaga

SEQ ID NO: 6 - DNA, 21 bp, Artificial Sequence
  Description of Artificial Sequence Primer
gagtcagaagaaagggcaaac

SEQ ID NO: 7 - DNA, 24 bp, Artificial Sequence
  Description of Artificial Sequence Primer
cagcgctgtcattgtgggaatggc

SEQ ID NO: 8 - DNA, 24 bp, Artificial Sequence
  Description of Artificial Sequence Primer
aggaaggcatctgcggcagtcacc